(19) United States
(12) Patent Application Publication
(10) Pub. No.: US 2015/0289803 A1
(43) Pub. Date: Oct. 15, 2015

(54) METHOD AND SYSTEM OF SLEEP DETECTION

(71) Applicant: Quanta Computer Inc., Kuei Shan Hsiang (TW)

(72) Inventors: Yu-Min Wu, Kuei Shan Hsiang (TW); Yung-Ming Chung, Kuei Shan Hsiang (TW); Yu-Siang Wang, Kuei Shan Hsiang (TW)

(21) Appl. No.: 14/447,741

(22) Filed: Jul. 31, 2014

(30) Foreign Application Priority Data
Apr. 14, 2014 (TW) 103113512

(51) Int. Cl. A61B 5/00; A61B 5/113; A61B 5/11; A61B 5/0205

(52) U.S. Cl. CPC A61B 5/4812; A61B 5/0205; A61B 5/1135; A61B 5/1118; A61B 5/4809; A61B 5/4866; A61B 5/0022; A61B 5/6823; A61B 5/7278; A61B 5/742; A61B 5/024

(57) ABSTRACT

A sleep detection system and method are provided. In the sleep detection system, a sensor device is configured to measure a heart rate of a user; a measuring device is configured to receive the heart rate, and measure an activity level of the user and calculate an energy-expenditure value according to the heart rate, the activity level and personal parameters of the user; and a receiving device is configured to receive the energy-expenditure value from the measuring device and generate a sleep analysis result according to the energy-expenditure and to display the sleep analysis result.

### CROSS REFERENCE TO RELATED APPLICATIONS

This Application claims priority of Taiwan Patent Application No. 103113512, filed on Apr. 14, 2014, the entirety of which is incorporated by reference herein.

### BACKGROUND OF THE INVENTION

1. Field of the Invention

The disclosure is related to sleep detection technology, and, more particularly, to analyzing the energy-expenditure value of the user.

2. Description of the Related Art

The sleep process of humans can be divided into two periods: a Slow Wave Sleep (SWS) period and a Fast Wave Sleep (FWS) period, as determined according to the varieties of the Electroencephalography (EEG), Electrooculography (EOG), and Electromyography (EMG) in the process. Slow Wave Sleep also can be regarded as a Deep Sleep or Non-rapid-eye-movement (NREM). Fast Wave Sleep also can be regarded as a Rapid-eye-movement (REM).

The SWS period can divided into four stages (Stage 1˜Stage 4), and every stage indicates different degree of sleep. In Stage 1, people doze off and can still feel external stimuli, such as numbness, trembling and other feelings. In this stage, there are still some cognitive abilities in the brain, and in the Electroencephalography, the α wave decreases and some θ waves appear (The spindle wave and K-complex wave usually don't appear at this stage. Even if the spindle wave or K-complex wave appear, the number of spindle waves or K-complex waves are not more than one per 1 second). In Stage 2, people already can't feel external stimuli and there are no cognitive abilities in the brain. The spindle waves, K-complex waves, and some δ waves appear in the Electroencephalography, wherein the percentage of δ waves is not more than 20%. In Stage 3, people move from the moderate sleep to the deep sleep, and more δ waves and some spindle waves appear in the Electroencephalography, wherein the percentage of δ waves is 20%-50%. In Stage 4, people are in deep sleep, and the percentage of δ waves is more than 50%.

Traditionally, the user may go to hospital to undergo polysomnography (PSG) detection to detect the sleep status of the user. Polysomnography detection comprises more detection items, such as Electroencephalography (EEG), Electrooculography (EOG), Electromyography (EMG), body position, Electrocardiography (ECG), and so on. In polysomnography detection, the user needs to accept the multi-sleep-physiology record in the hospital for recording the heart rate, blood oxygen level, breathing, brain waves, blood pressure and other status points of the user. The sleep performance and the sleep posture can be understood through the polysomnography detection. However, in polysomnography detection, the tester needs to sleep in the laboratory of the hospital, therefore, the tester who is having sleep problems undoubtedly would be affected by the strange environment and the result of the detection can be affected.

Therefore, a more convenient sleep detection method needs to be provided and the method is worth to discuss. In the method, the user doesn't need to go to hospital for the sleep detection by the complex detection apparatus, and can stay at home to do his sleep status analysis in an easier way.

### BRIEF SUMMARY OF THE INVENTION

A system and method of sleep detection are provided to overcome the problems mentioned above.

An embodiment of the invention provides a sleep-detection system. The sleep detection system comprises a sensor device which is configured to measure a heart rate of an user. The sleep detection system also comprises a measuring device which is configured to receive the heart rate, and measure an activity level of the user and calculate an energy-expenditure value according to the heart rate, the activity level and personal parameters of the user. The sleep detection system further comprises a receiving device which is configured to receive the energy-expenditure value from the measuring device and generate a sleep analysis result according to the energy-expenditure and to display the sleep analysis result.

An embodiment of the invention provides a sleep-detection method for a sleep detection system. The sleep detection method comprises the steps of measuring a heart rate of an user; measuring an activity level of the user; calculating an energy-expenditure value according to the heart rate, the activity level and personal parameters of the user; generating a sleep analysis result according to the energy-expenditure; and displaying the sleep analysis result.

Other aspects and features of the invention will become apparent to those with ordinary skill in the art upon review of the following descriptions of specific embodiments of communication transmission methods and systems

### BRIEF DESCRIPTION OF THE DRAWINGS

The invention will become more fully understood by referring to the following detailed description with reference to the accompanying drawings, wherein:

FIG. 1 is schematic diagram illustrating the sleep-detection system 100 according to an embodiment of the invention;

FIG. 2 is schematic diagram illustrating the measuring device 120 according to an embodiment of the invention;

FIG. 3 is schematic diagram illustrating the receiving device 130 according to an embodiment of the invention;

FIG. 4 is a flowchart 400 of a sleep detection method according to an embodiment of the invention;

FIG. 5 is a flowchart 500 of a sleep detection method according to another embodiment of the invention;

FIG. 6 is a flowchart 600 of a sleep detection method according to another embodiment of the invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

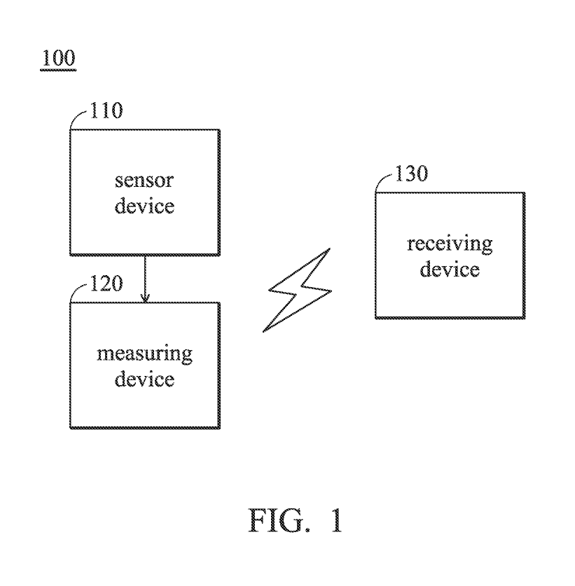

FIG. 1 is schematic diagram illustrating the sleep-detection system 100 according to an embodiment of the invention. As shown in FIG. 1, in an embodiment of the invention, the sleep-detection system 100 includes a sensor device 110, a measuring device 120 and a receiving device 130. Note that, in order to clarify the concept of the invention, FIG. 1 presents a simplified block diagram in which only the elements relevant to the invention are shown. However, the invention should not be limited to what is shown in FIG. 1 and the sleep-detection system 100 can further include other devices or elements. In an embodiment of the invention, the sensor device 110 and the measuring device 120 are placed on the user's body, wherein the measuring device 120 is placed on the center of the user's chest. The receiving device 130 is configured to receive the information from the measuring device 120 and display the information. In an embodiment of the invention, the sensor device 110 is combined in the measuring device 120.

In an embodiment of the invention, the sensor device 110 has one or plurality of measuring electrodes, such as an Electrocardiography (ECG) measuring electrode, and is connected to the measuring device 120. The sensor device 110 may measure the heart rate of the user in a sleeping state by the measuring electrodes and transmit a signal to the measuring device 120 according to the heart rate. When the measuring device 120 receives the signal, the measuring device 120 may obtain the heart rate of the user in a sleeping state. In an embodiment of the invention, the sensor device 110 may be regarded as a suit of clothes with measuring electrodes.

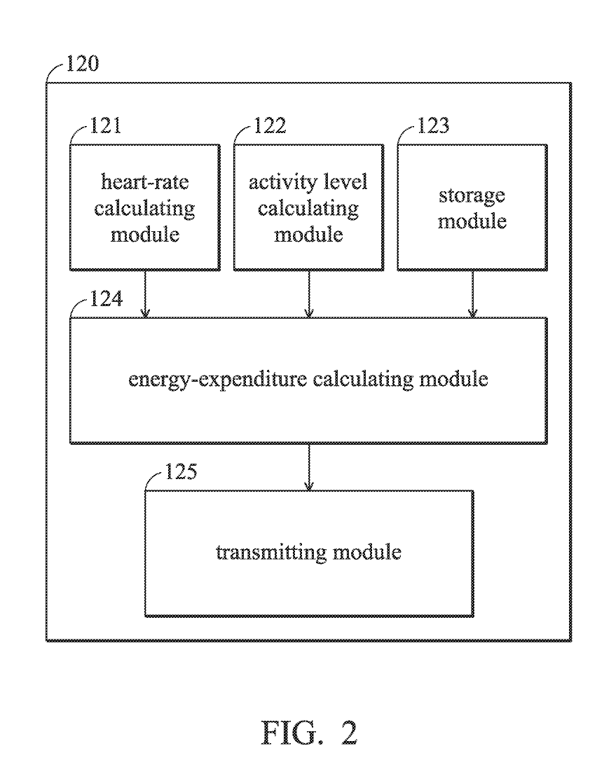

FIG. 2 is schematic diagram illustrating the measuring device 120 according to an embodiment of the invention. As shown in FIG. 2, the measuring device 120 includes a heart-rate calculating module 121, an activity level calculating module 122, a storage module 123, an energy-expenditure calculating module 124 and a transmitting module 125. When the sensor device 110 transmits the measured heart rate of the user in a sleeping state to the measuring device 120, the heart-rate calculating module 121 will receive the heart rate of the user in a sleeping state, and calculate the difference value between the heart rate of the user in a sleeping state and a preset heart rate in a sleeping state. In an embodiment of the invention, the preset heart rate in a sleeping state is defined as the average heart rate of the user in a normal state minus 10 (average heart rate-10).

In an embodiment of the invention, the activity level calculating module 122 is configured to detect the activity events of the user in a sleeping state by a plurality of sensors, such as g-sensor, and calculate the activity level of the user in a sleeping state according to the detected activity events of the user by an activity level algorithm. In an embodiment of the invention, the activity events of the user include the number of times the user turn over, the number of times the user moves, the number of times of the user's chest shakes, and so on. After the activity level calculating module 122 obtains the number of the activity events, it may calculate the activity level of the user according to the detected result of the detected activity events.

In an embodiment of the invention, when the heart-rate calculating module 121 has calculated the difference value and the activity level calculating module 122 has calculate the activity level of the user, the energy-expenditure calculating module 124 may calculate an energy-expenditure value according to the calculated results of the heart-rate calculating module 121 and the activity level calculating module 122 and the personal parameters of the user. In an embodiment of the invention, the personal parameters include the weight, height, sex and other parameters of the user. These personal parameters can be pre-input into the measuring device 120. In an embodiment of the invention, when the personal parameters are input into the measuring device 120, the personal parameters may be stored in the storage module 123.

In an embodiment of the invention, the energy-expenditure calculating module 124 calculates the energy-expenditure value by an energy-expenditure algorithm. In the energy-expenditure algorithm, first, the energy-expenditure calculating module 124 may determine whether the activity level is higher than a first threshold value, and whether the difference value of the heart rate of the user and a preset heart rate is higher than a second threshold value. Then, the energy-expenditure calculating module 124 may adopt different function coefficients to calculate the energy-expenditure intensity according to different determined results, such as, the activity level is higher or lower than the first threshold value and the difference value between the heart rate of the user and a preset heart rate is higher or lower than the second threshold. In an embodiment of the invention, the function is (A*calorific coefficient of the activity level+B*over-energy-expenditure coefficient), wherein the A and B are adjustable coefficients. The energy-expenditure calculating module 124 may adjust the value of A and B according to the different determined results. In an embodiment of the invention, the second threshold value may include a plurality of judgment ranges. That is to say, when the difference value of the heart rate of the user and a preset heart rate is compared with the second threshold value, if the difference value of the heart rate of the user and a preset heart rate is higher than a first value, the energy-expenditure calculating module 124 may execute a function; and if the difference value of the heart rate of the user and a preset heart rate is smaller than the first value the energy-expenditure calculating module 124 may determine whether the difference value of the heart rate of the user and a preset heart rate is higher than a second value and adopt the coefficient of the function according to the determined result. Note that while the first value and the second value have been described by way of example, it should be understood that the invention is not limited thereto. In some embodiments of the invention, higher judgment ranges may be adopted (e.g. third value, forth value and so on) according to different situations.

After calculating the energy-expenditure intensity, the energy-expenditure calculating module 124 may multiply the energy-expenditure intensity by the weight of the user to generate an energy-expenditure value. The variety of the user's energy expenditure (e.g. losing calories) in a sleeping state may be known via the energy-expenditure value.

The transmitting module 125 transmits the energy-expenditure value to the receiving device 130 by a wireless-communication transmission technology after the energy-expenditure calculating module 124 calculates the energy-expenditure value. In an embodiment of the invention, the wireless-communication transmission technology may be infrared ray, Bluetooth, 802.11 (Wi-Fi), ZigBee, Ultra WideBand, Near Field Communication (NFC) or another wireless-communication transmission technology.

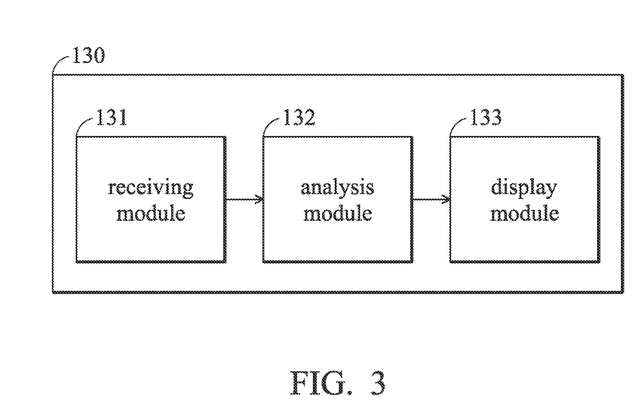

FIG. 3 is schematic diagram illustrating the receiving device 130 according to an embodiment of the invention. The receiving device 130 comprises a receiving module 131, an analysis module 132 and a display module 133. The receiving module 131 receives the energy-expenditure value from the transmitting module 125 of the measuring device 120 and transmits the energy-expenditure value to the analysis module 132. Then, the analysis module 132 may analyze the sleep status of the user according to the energy-expenditure value to generate a sleep analysis result. The analysis module 132 may divide the sleep process of the user into different stages, such as wake, rapid-eye-movement (REM), non-rapid-eye-movement (NREM) and so on according to the energy-expenditure value. In addition, the analysis module 132 may determine whether respiratory events occur in the user's sleep. When the energy-expenditure value varies greatly, the analysis module 132 may determine that respiratory events have occurred in the user's sleep and recode the number of the respiratory events.

The sleep analysis result is transmitted to the display module 133 after the analysis module 132 has analyzed the energy-expenditure value. The display module 133 may display the sleep analysis result of the user after receiving the sleep analysis result. For example, the display module 133 may display a structure diagram or display different interfaces corresponding to different items of the sleep analysis result to help the user understand and evaluate his sleep status according to the sleep analysis result. Therefore, the sleep status of the user is obtained in time by the display module 133, and the entire sleep status of the user also can be understood after the use wakes up.

In an embodiment of the invention, the sleep analysis result includes sleep period, respiratory events and sleep status. The sleep architecture of the user can be understood according to the sleep period. Namely, according to the sleep period, the length of the rapid-eye-movement (REM) period and non-rapid-eye-movement (NREM) period can be determined in one sleep period, as well as how much time each stage of the non-rapid-eye-movement (NREM) period occupy respectively in one sleep period. According to the respiratory events, the number of times hyperpnea or sleep apnea occurrs in the user's sleep can be known. The sleeping status of the user may be evaluated according to the analysis result of the sleep period and the respiratory events. The percentage of the rapid-eye-movement (REM) period and non-rapid-eye-movement (NREM) period in one sleep period or the percentage of the deep sleep state and light sleep state in one sleep period can be evaluated by the analysis result of the sleep period. The analysis result of the respiratory events is configured to determine whether the use exhibits the symptoms of somnipathy or the sleep disorder such as sleep apnea.

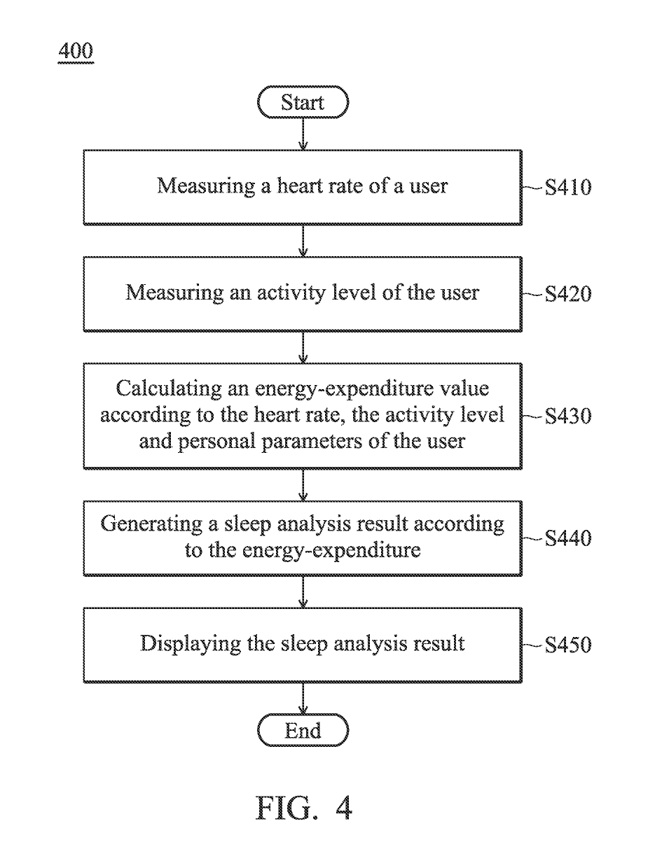

FIG. 4 is a flowchart 400 of a sleep detection method according to an embodiment of the invention. The method may be applied to the sleep-detection system 100. In step S410, the heart rate of a user is measured by the sensor device 110. In step S420, the activity level of the user is measured by the measuring device 120. In step S430, the energy-expenditure value of the user is calculated according to the heart rate, activity level, and personal parameters of the user by the measuring device 120. In step S440, a sleep analysis result is generated according to the energy-expenditure value by the receiving device 130. In step S450, the sleep analysis result is displayed by the receiving device 130.

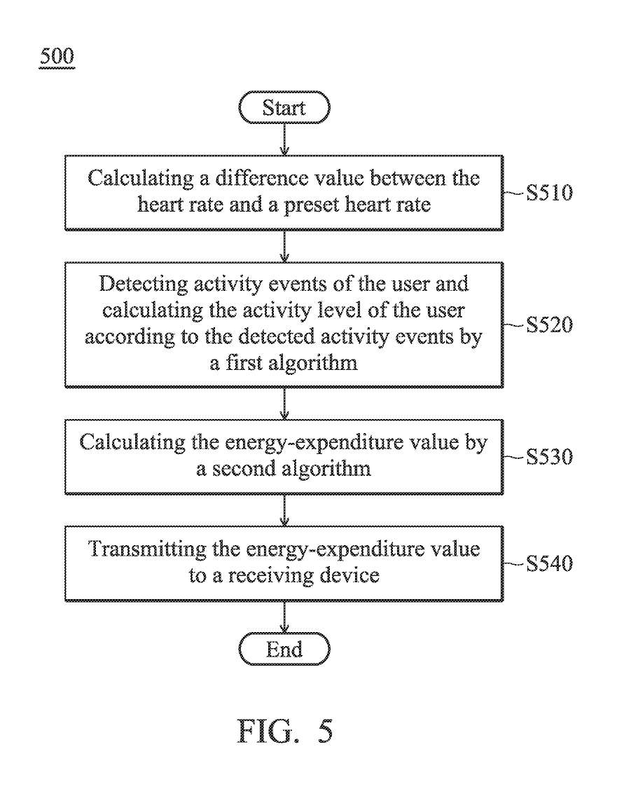

FIG. 5 is a flowchart 500 of a sleep detection method according to another embodiment of the invention. The method may be applied to the measuring device 120. In step S510, a difference value between the heart rate of the user and a preset heart rate is calculated by the measuring device 120. In step S520, a plurality of activity events of the user are detected by the measuring device 120, and the activity level of the user is calculate according to the detected activity events of the user by a first algorithm. In step S530, an energy-expenditure value is calculated by a second algorithm. In step S540, the energy-expenditure value is transmitted to a receiving device. In an embodiment of the invention, the first algorithm is an activity level algorithm, and the second algorithm is an energy-expenditure algorithm. In an embodiment of the invention, the activity events of the user includes the number of time the user turns over, the number of times the user moves, the number of times the user's chest shakes, and so on. In an embodiment of the invention, the personal parameters include the weight, height, sex and other parameters of the user.

In an embodiment of the invention, step S530 further includes determining whether the activity level is higher than a first threshold value, and whether the difference value between the heart rate of the user and a preset heart rate is higher than a second threshold value to calculate the energy-expenditure intensity. After calculating the energy-expenditure intensity, step S530 further includes multiplying the energy-expenditure intensity by the weight of the user to generate the energy-expenditure value.

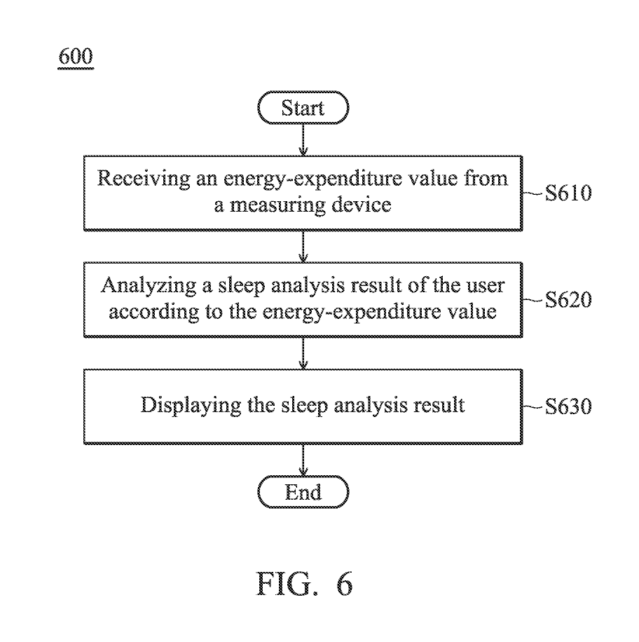

FIG. 6 is a flowchart 600 of a sleep detection method according to another embodiment of the invention. The method may be applied for the receiving device 130. In step S610, an energy-expenditure value is received from a measuring device by the receiving device 130. In step S620, a sleep analysis result of the user is analyzed according to the energy-expenditure value. In step S630, the sleep analysis result is displayed by the receiving device 130. In an embodiment of the invention, the sleep analysis result includes sleep period, respiratory events and sleep status.

The system and method of the sleep detection of the invention may be configured to analyze the sleep situation of the user according to the energy-expenditure value of the user. Therefore, the user doesn't need to go to hospital for polysomnography detection by the complex detection apparatus, and can stay at home to do his sleep status analysis by the system and method of the sleep detection of the invention.

The steps of the method described in connection with the aspects disclosed herein may be embodied directly in hardware, in a software module executed by a processor, or in a combination of the two. A software module (e.g., including executable instructions and related data) and other data may reside in a data memory such as RAM memory, flash memory, ROM memory, EPROM memory, EEPROM memory, registers, a hard disk, a removable disk, a CD-ROM, or any other form of computer-readable storage medium known in the art. A sample storage medium may be coupled to a machine such as, for example, a computer/processor (which may be referred to herein, for convenience, as a “processor”) such that the processor can read information (e.g., code) from and write information to the storage medium. A sample storage medium may be integral to the processor. The processor and the storage medium may reside in an ASIC. The ASIC may reside in user equipment. Alternatively, the processor and the storage medium may reside as discrete components in user equipment. Moreover, in some aspects any suitable computer-program product may comprise a computer-readable medium comprising codes relating to one or more of the aspects of the disclosure. In some aspects a computer program product may comprise packaging materials.

Reference throughout this specification to “one embodiment” or “an embodiment” means that a particular feature, structure, or characteristic described in connection with the embodiment is included in at least one embodiment of the invention, but does not denote that they are present in every embodiment. Thus, the appearance of the phrases “in one embodiment” or “in an embodiment” in various places throughout this specification are not necessarily referring to the same embodiment of the invention.

The above paragraphs describe many aspects of the invention. Obviously, the teaching of the invention can be accomplished by many methods, and any specific configurations or functions in the disclosed embodiments only present a representative condition. Those who are skilled in this technology can understand that all of the disclosed aspects in the invention can be applied independently or be incorporated.

While the invention has been described by way of example and in terms of preferred embodiment, it is to be understood that the invention is not limited thereto. Those who are skilled in this technology can still make various alterations and modifications without departing from the scope and spirit of this invention. Therefore, the scope of the present invention shall be defined and protected by the following claims and their equivalents.

What is claimed is:

1. A sleep detection system, comprising:
a sensor device, configured to measure a heart rate of a user;
a measuring device, configured to receive the heart rate, and measure an activity level of the user and calculate an energy-expenditure value according to the heart rate, the activity level and personal parameters of the user; and
a receiving device, configured to receive the energy-expenditure value from the measuring device and generate a sleep analysis result according to the energy-expenditure and to display the sleep analysis result.

2. The sleep detection system of claim 1, wherein the sensor device has one or a plurality of measuring electrodes to measure the heart rate.

3. The sleep detection system of claim 1, wherein the measuring device comprises:
a heart-rate calculating module, configured to calculate a difference value between the heart rate and a preset heart rate;
an activity level calculating module, configured to detect activity events of the user and calculate the activity level of the user according to the detected activity events by a first algorithm;
a storage module, configured to store the personal parameters;
an energy-expenditure calculating module, configured to calculate the energy-expenditure value according to the difference value, the activity level and the personal parameters by a second algorithm; and
a transmitting module, configured to transmit the energy-expenditure value to the receiving device by a wireless-communication transmission technology.

4. The sleep detection system of claim 3, wherein the activity events include the number of times the user turns over, the number of times the user moves, the number of times the user's chest shakes.

5. The sleep detection system of claim 3, wherein personal parameters include the weight, height, and sex of the user.

6. The sleep detection system of claim 3, wherein the second algorithm includes calculating an energy-expenditure intensity by determining whether the activity level is higher than a first threshold value, and whether the difference value is higher than a second threshold value and multiplying the energy-expenditure intensity by the weight of the user to generate the energy-expenditure value.

7. The sleep detection system of claim 6, wherein the second threshold value may include a plurality of judgment ranges.

8. The sleep detection system of claim 3, wherein the receiving device comprises:
a receiving module, configured to receive the energy-expenditure value by the wireless-communication transmission technology;
an analysis module, configured to analyze the energy-expenditure value to generate the sleep analysis result; and
a display module, configured to display the sleep analysis result.

9. The sleep detection system of claim 8, wherein the analysis module may divide a sleep process of the user into different stages according to the energy-expenditure value.

10. The sleep detection system of claim 8, wherein the sleep analysis result includes a sleep period, respiratory events and sleep status.

11. The sleep detection system of claim 1, wherein the measuring device is placed on the center of the user's chest.

12. A sleep detection method for a sleep detection system, comprising:
measuring a heart rate of a user;
measuring an activity level of the user;
calculating an energy-expenditure value according to the heart rate, the activity level and personal parameters of the user;
generating a sleep analysis result according to the energy-expenditure; and
displaying the sleep analysis result.

13. The sleep detection method of claim 12, further comprising:
calculating a difference value between the heart rate and a preset heart rate;
detecting activity events of the user and calculating the activity level of the user according to the detected activity events by a first algorithm; and
a storage module, configured to store the personal parameters; and
calculate the energy-expenditure value by a second algorithm.

14. The sleep detection method of claim 13, wherein the activity events include the number of times the user turns over, the number of times the user moves, the number of times the user's chest shakes.

15. The sleep detection method of claim 13, wherein personal parameters include the weight, height, and sex of the user.

16. The sleep detection method of claim 13, further comprising:
determining whether the activity level is higher than a first threshold value, and whether the difference value is higher than a second threshold value to calculate an energy-expenditure intensity; and
multiplying the energy-expenditure intensity by the weight of the user to generate the energy-expenditure value.

17. The sleep detection method of claim 16, wherein the second threshold value may include a plurality of judgment ranges.

18. The sleep detection method of claim 12, further comprising:
dividing a sleep process of the user into different stages according to the energy-expenditure value.

19. The sleep detection method of claim 12, wherein the sleep analysis result includes a sleep period, respiratory events and sleep status.
